(19) United States
(12) Patent Application Publication
(10) Pub. No.: US 2015/0203461 A1
(43) Pub. Date: Jul. 23, 2015

(54) PROCESSES FOR PRODUCING 5-(HYDROXYMETHYL)FURFURAL

(71) Applicant: E I DU PONT NEMOURS AND COMPANY, Wilmington, DE (US)

(72) Inventors: Subramaniam Sabesan, Wilmington, DE (US); Christina S. Stauffer, Wilmington, DE (US)

(21) Appl. No.: 14/353,778

(22) PCT Filed: Oct. 26, 2012
(86) PCT No.: PCT/US12/62314

§ 371 Date: Apr. 24, 2014

Related U.S. Application Data
(60) Provisional application No. 61/553,685, filed on Oct. 31, 2011.

(51) Int. Cl. C07D 307/48

(52) U.S. Cl. CPC C07D 307/48

(57) ABSTRACT

Processes for producing 5-(hydroxymethyl)furfural are provided. 5-(hydroxymethyl)furfural is a potential platform intermediate in the conversion of biomass to renewable chemicals. The processes use hexoses in an aqueous solution comprising a halide-containing salt, a water-miscible organic solvent, acid, and water providing an aqueous solution comprising a halide-containing salt, a water-miscible organic solvent, acid, and water. The use of small amounts of halide-containing salt results in significant increases in yield.

### FIELD OF THE INVENTION

Processes for producing 5-(hydroxymethyl)furfural from biomass are provided. The processes can produce 5-(hydroxymethyl)furfural from hexoses in the presence of halide-containing salts in an aqueous solution containing a water-miscible organic solvent.

### BACKGROUND OF THE INVENTION

Furfural, 5-(hydroxymethyl)furfural and related compounds are useful precursors and starting materials for industrial chemicals for use as pharmaceuticals, herbicides, stabilizers, and polymers. Conventionally, furfural can be produced from C₅ sugars which have been obtained from hydrolysis of the hemicellulose contained in biomass and 5-hydroxymethylfurfural can be produced from C₆ sugars which have been obtained from hydrolysis of the cellulose contained in biomass. Typically, the hydrolysis of biomass is performed with aqueous acids at relatively high temperatures to obtain C₅ and C₆ sugars derived from xylan and glucan, respectively.

In US Patent Publication No. 20110071306, Robinson discloses a method for the acid hydrolysis of carbohydrates in or from biomass, using a solvent system including an aqueous ether.

5-hydroxymethylfurfural (HMF) is a potential platform intermediate in the conversion of biomass to renewable chemicals. Despite the increasing interest in HMF as a platform chemical, a need remains for an economically viable commercial-scale dehydration process for HMF production.

### SUMMARY OF THE INVENTION

A process is provided comprising

a) providing an aqueous solution comprising a halide-containing salt, a water-miscible organic solvent, acid, and water;

b) providing a C₆ sugar

c) contacting the C₆ sugar with the aqueous solution to form a reaction mixture, wherein
i) the concentration of water-miscible organic solvent is from about 10 weight percent to about 99 weight percent,
ii) the concentration of halide-containing salt is from about 0.1 weight percent to about 2 weight percent,
iii) the acid is a homogeneous or heterogeneous acid, wherein the concentration of the homogeneous acid is from about 0.01 M to about 2 M, and the heterogeneous acid is from about 0.01 weight percent to about 30 weight percent, and
iv) the feedstock amount is from about 1 weight percent to about 50 weight percent,

wherein the weight percentages are based on the total weight of the reaction mixture;

and

d) heating the reaction mixture at a reaction temperature in the range of about 80° C. to about 190° C. for a time sufficient to effect a reaction to produce 5-(hydroxymethyl) furfural, wherein the aqueous solution is monophasic at the completion of the reaction.

### DETAILED DESCRIPTION

As used herein, where the indefinite article “a” or “an” is used with respect to a statement or description of the presence of a step in a process disclosed herein, unless the statement or description explicitly provides to the contrary, the use of such indefinite article does not limit the presence of the step in the process to one in number.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. The term “about” may mean within 10% of the reported numerical value, preferably within 5% of the reported numerical value.

As used herein, the term “biomass” refers to any hemicellulosic or lignocellulosic material and includes materials comprising hemicellulose, and optionally further comprising cellulose, lignin, starch, oligosaccharides and/or monosaccharides.

As used herein, the term “lignocellulosic” means, comprising both lignin and cellulose. Lignocellulosic material may also comprise hemicellulose. In some embodiments, lignocellulosic material contains glucan and xylan.

As used herein, the term “miscible” refers to a mixture of components that, when combined, form a single phase (i.e., the mixture is “monophasic”) under specified conditions (e.g., component concentrations, temperature).

As used herein, the term “monophasic” refers to a reaction medium that includes only one liquid phase. Some examples are water, aqueous solutions, and solutions containing aqueous and organic solvents that are miscible with each other. The term “monophasic” can also be used to describe a method employing such a reaction medium.

As used herein, the term “biphasic” refers to a reaction medium that includes two immiscible liquid phases, for example, an aqueous phase and a water-immiscible organic solvent phase. The term “biphasic” can also be used to describe a method employing such a reaction medium.

As used herein the term “water-miscible organic solvent” refers to an organic solvent that can form a monophasic solution with water at the temperature at which the reaction is carried out.

As used herein, the term “metal halide” refers to a compound generally described by the formula MXₙ, where M is a metal cation of valence+n, X is a halogen, and n ranges from +1 to +3.

Unless otherwise defined, all technical and scientific terms used herein have the same meaning as commonly understood by one of ordinary skill in the art to which this invention belongs. In case of conflict, the present specification including definitions, will control.

In the processes disclosed herein, one or more sugars is contacted with aqueous solution containing a water-miscible organic solvent, acid, and a salt containing an anion which is a halide (“halide-containing salt”) to produce a reaction mixture that, under suitable reaction conditions, produces a mixture comprising 5-(hydroxymethyl)furfural.

The sugar can be in solution, e.g., an aqueous solution. The sugar can be in a liquid solution or suspension of a biomass feedstock material, such as plant material.

The sugar is present in the reaction mixture at about 0.1 weight percent to about 50 weight percent based on the weight of the reaction mixture. In some embodiments, the sugar is present in the reaction mixture at a weight percentage between and optionally including any two of the following values: 0.1, 1, 5, 10, 15, 20, 25, 30, 35, 40, 45, and 50 weight percent.

Suitable sugars include monosaccharides, disaccharides, oligosaccharides, and polysaccharides, comprising C₆ sugar units (hexoses). The content of C₆ sugar units can be 100% of the total sugars, or less on a molar basis, such as 90, 80, 70, 60, 50, 40, 30, 20 or 10%. Preferred sugars are hexoses such as fructose, tagatose, sorbose, psicose, allose, altrose, glucose, mannose, gulose, idose, galactose and talose; disaccharides such as sucrose, maltose, lactose, cellobiose, and derivatives thereof; and polysaccharides such as maltodextrins, inulin, cellulose, starch, and derivatives thereof. In some embodiments, the sugars are obtained from lignocellulosic material, e.g., in a feedstock.

Suitable feedstocks include, for example, corn grain, corn cobs, corn husks, corn stover, grasses, wheat, wheat straw, barley, barley straw, hay, rice straw, switchgrass, waste paper, sugar cane bagasse, sorghum, soy, trees, branches, roots, leaves, wood chips, sawdust, shrubs, bushes, vegetables, fruits, flowers, and mixtures of any two or more thereof. Products and by-products from the milling of grains are also suitable.

The halide-containing salt is present in the reaction mixture at about 0.1 to about 2 weight percent based on the weight of the reaction mixture. In some embodiments, the halide-containing salt is present in the reaction mixture at a weight percentage between and optionally including any two of the following values: 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0 weight percent.

In some embodiments, the halide-containing salt is a metal halide. In some embodiments, the metal halide is a metal chloride, a metal bromide, or a metal iodide. In some embodiments, the metal halide is an alkali halide, an alkaline earth halide, or a transition metal halide. Mixtures of metal halides can also be used. In some embodiments, the metal halide is NaCl, NaI, NaBr, LiCl, LiI, LiBr, KCl, KBr, KI, MgCl₂, MgBr₂, MnCl₂, MnBr₂, RbCl, RbBr, ZnCl₂, ZnBr₂, BaCl₂, BaBr₂, CsCl, CsBr, CoCl₂, CoBr₂, CaCl₂, CaBr₂, NiCl₂, NiBr₂, CrCl₂, CrCl₃, CuCl, CuBr, CuI, CuCI₂, CuBr₂, AlCl₃, FeCl₂, FeBr₂, FeCl₃, FeBr₃, VCl₃, MoCl₃, LaCl₃, PdCl₂, PtCl₂, PtCl₄, RuCl₃, RhCl₃, or a mixture of any two or more of these. In some embodiments the metal halide is NaBr. It is desirable not to exceed an amount of metal halide at which the aqueous solution becomes biphasic.

In some embodiments, the halide-containing salt is an ionic liquid, which contains an anionic portion and a cationic portion. The anionic portion of the ionic liquid is a halide, including bromide, chloride and iodide. Suitable ionic liquids include members of the 1-R₁-3-R₂ imidazolium class of compounds, wherein R₁ and R₂ are alkyl groups containing from 1 to 10 carbons. Suitable imidazolium salts containing a halide anion are 1-ethyl-3-methylimidazolium chloride [EMIM]Cl and [EMIM]Br, and 1-butyl-3-methylimidazolium cloride [BMIM]Cl and [BMIM]Br. Other ionic liquids suitable for use include tetralkylammonium salts (e.g., N,N,N,N-tetraalkylammonium salts such as tetrabutylammonium bromide) and phosphonium salts (e.g., P,P,P,P-tetraalkylphosphonium salts) and pyridinium salts (e.g., N-alkylpyridinium salts) that include a stoichiometric amount of a suitable halide anion.

The acid can be a homogeneous or heterogeneous acid. Suitable homogeneous acids include mineral acids such as, for example, H₂SO₄, HCl, H₃PO₄, and HNO₃. In some embodiments, the acid is a heterogeneous acid. Suitable heterogeneous acids include zeolites (HY-zeolite, mordenite, faujasite, beta zeolite, aluminosilicates like MCM-20 and MCM-41, montmorillonite, and derivatives thereof), heteropolyacids (such as 12-tungstophosphoric acid, 12-molybdophosphoric acid, 12-tungstosilicic acid, 12-molybdosilicic acid and derivatives thereof), sulfated zirconias, solid metal phosphates (such as zirconium, titanium, niobium, and vanadyl phosphates), and acidic resins, such as ion-exchange resins (Amberlyst 15, Amberlyst 70, and DOWEX-type ion exchange resins and derivatives thereof).

When a homogeneous acid is used, it is present in the reaction mixture at about 0.01 M to about 2 M. In some embodiments, the homogeneous acid is present at about 0.1 M to 0.5 M, or about 0.1 M to 0.75 M, or about 0.1 M to 1 M. In some embodiments, the homogeneous acid is present at a molarity between and optionally including any two of the following values: 0.01 M, 0.05 M, 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M, 1.0 M, 1.5 M, and 2.0 M.

When a heterogeneous acid is used, it is present in the reaction mixture at about 0.01 weight percent to about 30 weight percent based on the weight of the reaction mixture. In some embodiments, the heterogeneous acid is present in the reaction mixture at a weight percentage between and optionally including any two of the following values: 0.01, 0.05, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 15, 20, 25, and 30 weight percent.

The water-miscible organic solvent is present in the reaction mixture at about 10 weight percent to about 99 weight percent based on the weight of the reaction mixture. In some embodiments, the water-miscible organic solvent is present in the reaction mixture at about 50 weight percent to about 95 weight percent, or about 70 weight percent to about 95 weight percent. In some embodiments, the water-miscible organic solvent is present in the reaction mixture at a weight percentage between and optionally including any two of the following values: 10, 15, 20, 25, 30, 35, 40, 45, 50, 55, 60, 65, 70, 75, 80, 85, 90, 91, 92, 93, 94 and 95 weight percent. In some embodiments the water-miscible organic solvent is an ether. Examples of suitable ethers include: tetrahydrofuran (“THF”), dioxane, 2-methoxy-methylethoxy-propanol, and dimethyl ether. Mixtures of ethers can also be used. In some embodiments, the ether is THF. In other embodiments, the water-miscible organic solvent is acetonitrile, acetone, methanol, ethanol, isopropanol, dimethylsulfoxide, N,N-dimethylformamide, N,N-dimethylacetamide, sulfolane, diethyleneglycol, and ethylene glycol.

The reaction mixture is heated at a reaction temperature within the range from about 80° C. to about 190° C. for a time sufficient to effect a reaction to produce 5-(hydroxymethyl) furfural. In some embodiments, the reaction mixture remains monophasic at the reaction temperature and is monophasic at the conclusion of the reaction. In some embodiments, the reaction mixture is biphasic at the reaction temperature and is monophasic at the conclusion of the reaction. The period of time for heating is within the range of about 1 minute to about 10 hours. In some embodiments, the temperature is between and optionally including any two of the following values: 80, 85, 90, 95, 100, 105, 110, 115, 120, 125, 130, 135, 140, 145, 150, 155, 160, 165, 170, 175, 180, 185, and 190° C. In some embodiments, the reaction mixture is heated at a temperature within the range from about 110° C. to about 140° C. The appropriate temperature varies, depending on factors including type of feedstock, feedstock particle size, and component concentrations, and is readily determined by one of ordinary skill in the art.

In some embodiments, the reaction mixture is heated for a period of time between and optionally including any two of the following values: 1 min, 5 min, 10 min, 0.25 h, 0.33 h, 0.42 h, 0.5 h, 0.75 h, 1 h, 2 h, 3 h, 4 h, 5 h, 6 h, 7 h, 8 h, 9 h, and 10 h. In some embodiments, the reaction mixture is heated for about 5 minutes to about 4 hours. The appropriate amount of time varies, depending upon conditions such as temperature, type of feedstock, feedstock particle size, component concentrations; and is readily determined by one of ordinary skill in the art.

In some embodiments, the reaction mixture is pressurized under an inert gas. Suitable inert gases include nitrogen and argon. The reaction is pressurized between and optionally including any two of the following values: 25 psi, 50 psi, 100 psi, 150 psi, 200 psi, 250 psi, 300 psi, 350 psi, 400 psi, 450 psi, and 500 psi. In some embodiments, the reaction mixture is pressurized at 400 psi. In some embodiments, the reaction is run under autogenous pressure.

The processes disclosed herein can be performed in any suitable vessel, such as a batch reactor or a continuous reactor. The suitable vessel may be equipped with a means, such as impellers, for agitating the reaction mixture. Reactor design is discussed, for example, by Lin, K.-H., and Van Ness, H. C. (in Perry, R. H. and Chilton, C. H. (eds.), Chemical Engineer's Handbook, 5th Edition (1973) Chapter 4, McGraw-Hill, NY). The contacting step may be carried out as a batch process, or as a continuous process.

After the reaction mixture has been heated for the appropriate period of time as recited above, the HMF thereby produced is recovered by an appropriate method known in the art, such as, for example, chromatography, extraction, distillation, adsorption by resins, separation by molecular sieves, or pervaporation. In some embodiments, distillation is used to recover the HMF from the reaction mixture. In some embodiments, extraction is used to recover the HMF from the reaction mixture.

### Examples

The methods disclosed herein are illustrated in the following examples. From the above discussion and these examples, one skilled in the art can ascertain the essential characteristics of this invention, and without departing from the spirit and scope thereof, can make various changes and modifications of the invention to adapt it to various uses and conditions.

All commercial reagents were used as received. All chemicals were obtained from Sigma-Aldrich (St. Louis, Mo.) unless stated otherwise. Amberlyst 70 was obtained from Rohm and Hass (Midland, Mich.), tetrahydrofuran and NaCl were obtained from EMD Chemicals (Gibbstown, N.J.), and 1-Butyl-3-methylimidazolium bromide ([BMIM]Br) was purchased from Acros (Geel, Belgium).

The following abbreviations are used in the examples: “° C.” means degrees Celsius; “RPM” means revolutions per minute; “wt %” means weight percent; “g” means gram; “min” means minute(s); “μL” means microliter; “wt %” means weight percent; “RV(s)” means reaction vessel(s); “PSI” means pounds per square inch; “mg/g” means milligram per gram; means micrometer; “mL” means milliliter; “mm” means millimeter and “mL/min” means milliliter per minute; “THF” means tetrahydrofuran; “HMF” means 5-hydroxymethylfurfural; “MPa” means megapascal.

### GENERAL METHODS

### Sample Preparation and Reaction Conditions

All reactions were performed in an Endeavor Parallel Pressure Reactor (Biotage LLC, Charlotte, N.C., USA). The glass reaction vessel (RV) inserts were prepared for each sample as specified with a total weight of 5 g. The reaction vessels were loaded into the reactor block (up to eight at a time) and the stirring was set at 450 RPM. The RVs were purged with nitrogen three times, pressurized to 100 psi (0.69 MPa) and then heated to the specified reaction temperature over a period of 15 min. The pressure was then increased to 400 psi (2.76 MPa) and the RVs were left in this state for the specified time before heating was shut off and the RVs were left to cool below 50° C. The RVs were then removed from the reactor block and the samples were transferred to glass vials where they were diluted with water for a total mass of 14-20 g. All reaction samples were then analyzed for the presence of HMF using HPLC.

### HPLC Analysis of Reaction Samples

Each cooled reaction sample was transferred to a glass vial and diluted with water to a mass between 14-20 g. One gram of the diluted reaction sample was then added to a second glass vial. To this second vial was added one gram of 2-hexanol in distilled water (5 mg/g) as the internal standard. A solution of 1% sodium bicarbonate in water was also added to the vial to bring the sample weight up to 5 g. The sample was mixed thoroughly and 1 mL was then filtered through a 0.2 μm filter (GHP Acrodisc 13 mm syringe filter, PALL Life Sciences, Port Washington, N.Y.). The soluble products in the reaction mixture, namely hexoses (glucose, fructose) and HMF were measured by HPLC (1200 Series, Agilent Technologies, Santa Clara, Calif.) using an Aminex HPX-87P column (300 mm×7.8 mm, Bio-Rad Laboratories, Hercules, Calif.) fitted with a guard column and detected using a RI detector. The column and guard column were held at 80° C. and the RI Detector was held at 55° C. Injection volume was 20 μL and sample run times were 60 minutes in length with a 0.6 mL/min flow rate using a water mobile phase. Concentrations were determined from a standard calibration curved developed for each of the analytes with 2-hexanol. Retention time of HMF using this HPLC method was 35.4 min.

### Example 1

### Synthesis of HMF from Fructose Using Aqueous THF with and without 1% NaBr

Fructose (0.25 g, 5 wt %) was loaded into the reaction vessels with Amberlyst 70 (0.25 g) and sodium bromide (0.05 g, 1 wt %). The solvent was 92% THF in water and was added in the appropriate amount to bring the reaction sample to a total of 5 g. All reactions samples were heated to 130° C. for 20 minutes as described in the General Methods, except for sample 34-5 which was heated for 30 mins. Upon completion, the reaction samples were analyzed via HPLC as described in the General Methods. As shown in Table 1, HMF yields were higher when the dehydration reaction was run in aqueous THF solvent containing sodium bromide.

TABLE #1

| | Reaction | NaBr | % Yield |
|---|---|---|---|
| Sample | Conditions | loading | HMF |
| 34-5 | 5 wt % Fructose, | 0% | 59 |
| 49-1 | 0.25 g Amberlyst 70, | 0% | 70 |
| 39-3 | 130° C., 20 min | 1% | 81 |
| 49-3 | (except 30 min for | 1% | 79 |
| | sample 34-5) | | |

### Example 2

### Synthesis of HMF from Fructose Using 100% Water with 1% NaBr Versus Aqueous THF with 1% NaBr

Fructose (0.25 g, 5 wt %) was loaded into the reaction vessels with Amberlyst 70 (0.25 g) and sodium bromide (0.05 g, 1 wt %, if required). The solvent was either 100% water or 90% THF in water and was added in the appropriate amount to bring the reaction sample to a total of 5 g. Reactions were run in duplicate. All reactions samples were heated to 130° C. for 20 minutes as described in the General Methods. Upon completion, the reaction samples were analyzed via HPLC as described in the General Methods. As shown in Table 2, HMF yields were higher when run in aqueous THF solvent containing sodium bromide.

TABLE #2

| | Reaction | Solvent | NaBr | % Yield |
|---|---|---|---|---|
| Sample | Conditions | System | loading | HMF |
| 53-1 | 5 wt % Fructose, | 100% water | 1% | 32 |
| 53-2 | 0.25 g Amberlyst | | 1% | 26 |
| 53-5 | 70, | 90% THF in | 1% | 73 |
| 53-6 | 130° C., 20 min | water | 1% | 72 |

### Example 3

### Synthesis of HMF from Fructose in Aqueous THF Varying the Halide Salt

Fructose (0.25 g, 5 wt %) was placed in a reaction vessel, along with Amberlyst 70 (0.25 g), halide salt (0.05 g, 1 wt %), water (0.35 g), and THF (4.1 g). All reactions samples were heated to 130° C. for 20 minutes as described in the General Methods, Upon completion, the reaction samples were analyzed via HPLC as described in the General Methods. The reaction without any salt was carried out at two different temperatures and the sample containing NaBr was carried out in duplicate. As shown in Table 3, HMF yield was higher in the presence of a salt with a halide counteranion versus the samples that did not contain a halide salt.

TABLE #3

| | Reaction | | % HMF |
|---|---|---|---|
| Sample | Conditions | Salt | Yield |
| 34-5 | 30 min | No salt | 59 |
| 49-1 | 5 wt % Fructose, | No salt | 70 |
| 39-1 | 0.25 g Amberlyst 70, | NaCl | 77 |
| 39-3 | 1 wt % salt, 92% THF | NaBr | 81 |
| 49-3 | in water | NaBr | 79 |
| 39-8 | 130° C., 20 min | KBr | 71 |
| 40-2 | | Tetrabutylammonium | 74 |
| | | Bromide | |
| 40-4 | | [BMIM]Cl | 74 |
| 40-5 | | [BMIM]Br | 76 |
| 40-6 | | [EMIM]Cl | 76 |

### Example 4

### Examination of Varying the THF Concentration in Water on the Yield of HMF from Fructose with 1 wt % NaBr

Fructose (0.25 g, 5 wt %) was loaded into the vials in the presence of Amberlyst 70 (0.25 g), sodium bromide (0.05 g, 1 wt %) and a solvent system composed of THF in water (0-100%). The vials were heated to 130° C. for 20 minutes as described in the General Methods and all the reaction samples were prepared for analysis by HPLC as described in the General Methods. As the percentage of THF in water increased, the yield of HMF also increased (Table 4).

TABLE #4

| | Reaction | % THF in | % Yield |
|---|---|---|---|
| Sample | Conditions | Water | HMF |
| 52-1 | 0.25 g Fructose, | 0 | 32 |
| 52-2 | 0.25 g Amberlyst 70, | 10 | 33 |
| 52-3 | 1 wt % NaBr, | 30 | 37 |
| 52-4 | 130° C., 20 min | 50 | 47 |
| 52-5 | | 70 | 75 |
| 52-6 | | 80 | 73 |
| 52-7 | | 90 | 76 |
| 52-8 | | 100 | 64 |

### Example 5

### Examination of Varying the wt % Halide Salt Loading on the Synthesis of HMF from Fructose

Fructose (0.25 g) was loaded into reaction vessels along with Amberlyst 70 (0.25 g) and a solvent system composed of 70% THF in water. Sodium bromide was added in amounts between 1-1.5 wt %. The reaction samples were heated to 130° C. for 20 min according to the procedure described in the General Methods. Samples were run in duplicate. The reaction samples were analyzed by HPLC as described in the General Method. Samples 54-1 through 54-4 were monophasic at the start of the reaction and samples 54-5 and -6 were initially biphasic. As shown in Table 5, the HMF yield remained approximately the same for 1-1.5% NaBr added to the reaction samples.

TABLE #5

| | Reaction | NaBr | % Yield |
|---|---|---|---|
| Sample | Conditions | (wt %) | HMF |
| 54-1 | 0.25 g fructose, | 1 | 76 |
| 54-2 | 0.25 g Amberlyst 70, | 1 | 75 |
| 54-3 | 70% THF in water, | 1.25 | 73 |
| 54-4 | 130° C., 20 min | 1.25 | 75 |
| 54-5 | | 1.5 | 75 |
| 54-6 | | 1.5 | 74 |

### Example 6

### Comparison of HMF Yield from Fructose at 10 wt % Sugar Loading with Varying the Fructose:Resin Ratio—in Either 100% Water or 90% THF in Water, with or without 1% NaBr

Fructose at 10 wt % loading was placed into reaction vessels along with Amberlyst 70 (at a 1:1 or 1:2 wt % ratio compared to fructose), NaBr (0.05 g), and either 100% water or 90% THF in water as the solvent. The reactions samples were heated to 130° C. for 20 min according to the procedure described in the General Methods. The reaction samples were analyzed by HPLC as described in the General Method. At both 1:1 and 2:1 fructose to resin ratios, the HMF yield was higher in the aqueous THF solvent system compared to the HMF yield in 100% water (Table 6). The HMF yield was the highest in aqueous THF with NaBr with a higher fructose:resin ratio (compare 55-1 versus 55-10).

TABLE #6

| | Reaction | Fructose: | Solvent | | % Yield |
|---|---|---|---|---|---|
| Sample | Conditions | resin | System | NaBr | HMF |
| 55-6 | 10 wt % | 1:1 | Water | 0% | 23 |
| 55-5 | sugar | | Water | 1% | 24 |
| 55-2 | 130° C., | | 90% THF in water | 0% | 52 |
| 55-1 | 20 min | | 90% THF in water | 1% | 58 |
| 55-13 | | 2:1 | Water | 0% | 23 |
| 55-12 | | | Water | 1% | 36 |
| 55-11 | | | 90% THF in water | 0% | 57 |
| 55-10 | | | 90% THF in water | 1% | 76 |

### Example 7

### Comparison of HMF Yield from Fructose at 30 wt % Sugar Loading with Varying the Fructose:Resin Ratio—in Either 100% Water or 90% THF in Water, with or without 1% NaBr

Fructose at 30 wt % loading was placed into reaction vessels along with Amberlyst 70 (at a 1:1 or 1:6 wt % ratio compared to fructose), NaBr (0.05 g), and either 100% water or 90% THF in water as the solvent. The reactions samples were heated to 130° C. for 20 min according to the procedure described in the General Methods. The reaction samples were analyzed by HPLC as described in the General Method. At both 1:1 and 6:1 fructose to resin ratios, the HMF yield was higher in the aqueous THF solvent system compared to the HMF yield in 100% water (Table 7). The HMF yield was the highest in aqueous THF with NaBr with a higher fructose:resin ratio (compare 55-3 versus 55-8).

TABLE #7

| | Reaction | Fructose: | Solvent | | % Yield |
|---|---|---|---|---|---|
| Sample | Conditions | resin | System | NaBr | HMF |
| 55-9 | 30 wt % | 1:1 | Water | 0% | 4 |
| 55-7 | sugar | | Water | 1% | 9 |
| 55-4 | 130° C., | | 90% THF in water | 0% | 10 |
| 55-3 | 20 min | | 90% THF in water | 1% | 12 |
| 55-16 | | 6:1 | Water | 0% | 18 |
| 55-15 | | | Water | 1% | 25 |
| 55-14 | | | 90% THF in water | 0% | 17 |
| 55-8 | | | 90% THF in water | 1% | 48 |

### Example 8

### HMF Formation from Fructose Using Various Acids in Aqueous THF with and without 1 wt % NaBr

Fructose (0.25 g) was loaded into the reaction vessels along with acid (HCl, H₂SO₄, or H₃PO₄), 100% water or 90% THF in water as solvent, and 1 wt % sodium bromide, if required. All reaction samples were heated to 130° C. for 20 minutes as described in the General Methods. Upon completion, the reaction mixtures were analyzed using HPLC as described in the General Methods. All samples were run in duplicate. As shown in Table 8, HMF was formed in the presence of various adds. The HMF yields were highest in aqueous THF containing 1 wt % NaBr.

TABLE #8

| | | | | | HMF |
|---|---|---|---|---|---|
| | Reaction | Solvent | Acid | Wt % | Yield |
| Sample | Conditions | System | Concentration | NaBr | (%) |
| 58-2 | 0.25 g | 90% | 0.15M HCl | 0% | 57.5 |
| 58-4 | fructose, | THF | | | 54.8 |
| 58-1 | 130° C., 20 min | in water | | 1% | 65.9 |
| 58-3 | | | | | 75.3 |
| 58-6 | | | 0.15M H₂SO₄ | 0% | 61.6 |
| 58-8 | | | | | 53.8 |
| 58-5 | | | | 1% | 67.0 |
| 58-7 | | | | | 65.1 |
| 58-10 | | | 0.25M H₃PO₄ | 0% | 13.3 |
| 58-12 | | | | | 4.8 |
| 58-9 | | | | 1% | 46.9 |
| 58-11 | | | | | 35.9 |
| 58-18 | | 100% | 0.25M H₃PO₄ | 0% | 13.3 |
| 58-20 | | water | | | 14.6 |
| 58-17 | | | | 1% | 17.4 |
| 58-19 | | | | | 13.2 |

### Example 9

### Examination of HMF Formation from Fructose in Various Solvents—with and without NaBr

The reaction vessels were loaded with fructose (0.25 g), Amberlyst 70 (0.25 g), 90% 1,4-dioxane or acetonitrile in water, and NaBr, if required. The reaction vessels were heated to 130° C. for 20 minutes as described in the General Methods. Upon completion, the reaction mixtures were analyzed using HPLC as described in the General Methods. As seen in Table #9, the HMF yield was higher in both 1,4-dioxane and acetonitrile in the presence of NaBr.

TABLE #9

| | Reaction | Solvent | Wt % | HMF |
|---|---|---|---|---|
| Sample | Conditions | System | NaBr | Yield (%) |
| 61-1 | 0.25 g fructose | 90% 1,4-dioxane | 0% | 53.7 |
| 61-2 | 0.25 g Amberlyst 70, | in water | 1% | 66.3 |
| 61-3 | 130° C., 20 min | 90% acetonitrile | 0% | 37.9 |
| 61-4 | | in water | 1% | 77.3 |

### Example 10

### Examination of HMF Formation from Fructose in Aqueous THF while Varying the Wt % Loading of NaBr

The reaction vessels were loaded with 0.25 g fructose, 0.25 g Amberlyst 70, and the desired amount of sodium bromide in 90% THF in water, up to a total of 5 g. The reaction vessels were heated up to 130° C. for 20 minutes as described in the General Methods. Upon completion, the vials were analyzed using HPLC as described in the General Methods. The samples were run in duplicate and the HMF yields were averaged. As shown in Table #10, the highest HMF yield was seen with 1 wt % loading NaBr.

TABLE #10

| | Reaction | Solvent | Wt % | HMF |
|---|---|---|---|---|
| Sample | Conditions | System | NaBr | Yield (%) |
| 64-1, 2 | 0.25 g Fructose, | 90% THF | 0% | 64.9 |
| 64-3, 4 | 0.25 g Amberlyst 70, | in water | 0.1% | 68.6 |
| 64-5, 6 | 130° C., 20 min. | | 0.5% | 74.3 |
| 39-3, | | | 1% | 80.0 |
| 49-3 | | | | |

### Example 11

### Time and Temperature Optimization of HMF Formation from Fructose Using Amberlyst 70 and 1 wt % NaBr in Aqueous THF

The reaction vessels were loaded with 0.25 g fructose, 0.25 g Amberlyst 70, and 1 wt % sodium bromide in 90% THF in water. The reaction vessels were heated to the desired temperature for the designated amount of time as described in the General Methods. They were analyzed using HPLC as described in the General Methods. The results of this experiment are shown in Table #11.

TABLE #11

| | Reaction | Temperature | Time | HMF |
|---|---|---|---|---|
| Sample | Conditions | (° C.) | (min) | Yield (%) |
| 63-1 | 0.25 g fructose, | 100 | 10 | 24.6 |
| 63-2 | 0.25 g Amberlyst 70, | | 20 | 31.5 |
| 63-3 | 1 wt % NaBr, | | 30 | 41.2 |
| 63-4 | 90% THF in water | 110 | 10 | 53.9 |
| 63-5 | | | 20 | 64.0 |
| 63-6 | | | 30 | 71.2 |
| 63-7 | | 130 | 10 | 71.5 |
| 52-7 | | | 20 | 76.5 |
| 63-9 | | | 30 | 68.4 |
| 59-4 | | 150 | 10 | 64.0 |
| 59-8 | | | 20 | 60.5 |
| 63-12 | | | 30 | 47.1 |

### Example 12

### Examination of HMF Formation from Various Sources of C6 Sugars in Aqueous THF or Water, with and without 1 wt % NaBr

The reaction vessels were loaded with 0.25 g sugar, 0.25 g Amberlyst 70, and 1 wt % NaBr, in 90% THF in water. The samples were heated up to 130° C. for 20 minutes as described in the General Methods. After the samples were cooled to room temperature, the reaction mixtures were analyzed using HPLC as described in the General Methods. As shown in Table #12, it can be seen that different sources of C6 sugars provide a higher HMF yield in the presence of NaBr in aqueous THF.

TABLE #12

| | Reaction | | Solvent | Wt % | HMF |
|---|---|---|---|---|---|
| Sample | Conditions | Sugar | System | NaBr | Yield (%) |
| 56-1 | 0.25 g sugar, | Sucrose | Water | 0% | 1.0 |
| 56-2 | 0.25 g | | | 1% | 11.2 |
| 56-3 | Amberlyst 70, | | 90% THF in | 0% | 31.3 |
| 56-4 | 130° C., 20 min | | water | 1% | 39.9 |
| 56-5 | | Inulin | Water | 0% | 18.9 |
| 56-6 | | | | 1% | 34.2 |
| 56-7 | | | 90% THF in | 0% | 76.1 |
| 56-8 | | | water | 1% | 81.4 |
| 62-5 | | Sorbose | 90% THF in | 0% | 46.2 |
| 62-6 | | | water | 1% | 53.3 |

What is claimed is:

1. A process comprising:
a) providing an aqueous solution comprising a halide-containing salt, a water-miscible organic solvent, acid, and water;
b) providing a C₆ sugar
c) contacting the C₆ sugar with the aqueous solution to form a reaction mixture, wherein
i) the concentration of water-miscible organic solvent is from about 10 weight percent to about 99 weight percent,
ii) the concentration of halide-containing salt is from about 0.1 weight percent to about 2 weight percent,
iii) the acid is a homogeneous or heterogeneous acid,
wherein the concentration of the homogeneous acid is from about 0.01 M to about 2 M, and the heterogeneous acid is from about 0.01 weight percent to about 30 weight percent, and
iv) the feedstock amount is from about 1 weight percent to about 50 weight percent,
wherein the weight percentages are based on the total weight of the reaction mixture;
and
d) heating the reaction mixture at a reaction temperature in the range of about 80° C. to about 190° C. for a time sufficient to effect a reaction to produce 5-(hydroxymethyl) furfural, wherein the aqueous solution is monophasic at the completion of the reaction.

2. The process of claim 1 wherein the halide-containing salt is a metal halide.

3. The process of claim 2 wherein the metal halide is NaCl, NaBr, NaI, KCl, KBr, KI, LiCl, LiBr, LiI, RbCl, RbBr, CsCl, CsBr, or a mixture of any two or more thereof.

4. The process of claim 1 wherein the halide-containing salt is an alkaline earth halide.

5. The process of claim 4 wherein the alkaline earth halide is CaCl₂, CaBr₂, MgCl₂, MgBr₂, or a mixture of any two thereof.

6. The process of claim 1 wherein the halide-containing salt is a transition metal halide.

7. The process of claim 6 wherein the transition metal halide is NiCl₂, NiBr₂, CrCl₂, CrCl₃, CuCl, CuBr, CuI, CuCl₂, CuBr₂, AlCl₃, FeCl₂, FeBr₂, FeCl₃, FeBr₃, MnCl₂, MnBr₂, ZnCl₂, ZnBr₂, CoCl₂, CoBr₂, or a mixture of any two thereof.

8. The process of claim 1 wherein the halide-containing salt is an ionic liquid.

9. The process of claim 9 wherein the ionic liquid is selected from the group consisting of 1-ethyl-3-methylimidazolium chloride, 1-ethyl-3-methylimidazolium bromide, 1-butyl-3-methylimidazolium chloride, 1-butyl-3-methylimidazolium bromide, tetrabutylammonium chloride, and tetrabutylammonium bromide.

10. The process of claim 1 wherein the acid is selected from the group consisting of HCl, H₂SO₄, H₃PO₄, HNO₃, acidic ion-exchange resins, such as Amberlyst 15, Amberlyst 70, and DOWEX-type ion-exchange resins, zeolites, heteropolyacids, sulfated zirconias, and solid metal phosphates.

11. The process of claim 1 wherein the water-miscible organic solvent is an ether.

12. The process of claim 11 wherein the ether is THF or 1,4-dioxane.

13. The process of claim 11 wherein the ether is present at a quantity from about 50 weight percent to about 99 weight percent of the reaction mixture.

14. The process of claim 1 wherein the feedstock is selected from the group consisting of: corn grain, corn cobs, corn husks, corn stover, grasses, wheat, wheat straw, barley, barley straw, hay, rice straw, switchgrass, waste paper, sugar cane bagasse, sorghum, soy, trees, branches, roots, leaves, wood chips, sawdust, shrubs, bushes, vegetables, fruits, flowers, and mixtures of any two or more thereof.

15. The process of claim 1 wherein the reaction mixture is pressurized under an inert gas from about 25 psi (0.17 MPa) to about 500 psi (3.45 MPa).

16. The process of claim 1 wherein the reaction is run under autogenous pressure.

17. The process of claim 1 wherein the C₆ sugar is contained in a feedstock.

18. The process of claim 17 wherein the feedstock comprises at least one selected from the group consisting of:
i) lignocellulosic feedstock,
ii) cellulosic material, and
iii) C₆ sugar monomers, disaccharides, oligosaccharides and polysaccharides;
